(19) United States
(12) Patent Application Publication
(10) Pub. No.: US 2012/0329061 A1
(43) Pub. Date: Dec. 27, 2012

(54) QUANTITATIVE RT-PCR DETECTION FOR GENES INVOLVED IN EPITHELIAL MESENCHYMAL TRANSITION IN PERIPHERAL BLOOD OF CANCER PATIENTS

(75) Inventors: Michal Mego, Sturovo (SK); Sendurai Mani, Houston, TX (US); Massimo Cristofanilli, Philadelphia, PA (US); Naoto T. Ueno, Houston, TX (US); James M. Reuben, Houston, TX (US)

(73) Assignee: BOARD OF REGENTS, THE UNIVERSITY OF TEXAS SYSTEM, Austin, TX (US)

(21) Appl. No.: 13/583,378

(22) PCT Filed: Mar. 11, 2011
(86) PCT No.: PCT/US11/28049

§ 371 Date: Sep. 7, 2012

Related U.S. Application Data
(60) Provisional application No. 61/312,971, filed on Mar. 11, 2010.

(51) Int. Cl. C12Q 1/68

(52) U.S. Cl. 435/6.12

(57) ABSTRACT

An assay and associated methodologies for detection of breast cancer are described. Circulating tumor cells (“CTCs”) undergo epithelial mesenchymal transition (“EMT”) prior to entering circulation, resulting in the loss of epithelial markers. These CTCs and EMT-related gene transcripts in the peripheral blood of patients are tested by quantitative RT-PCR to detect and diagnose breast cancer.

### CROSS-REFERENCE TO RELATED APPLICATIONS

This application claims priority to U.S. Pat. App. Ser. No. 61/312,971 filed Mar. 11, 2010. The application is incorporated by reference herein in its entirety.

### FIELD OF THE INVENTION

The present invention is directed to quantitative RT-PCR detection of genes involved in epithelial mesenchymal transition in peripheral blood of cancer patients to determine the reoccurrence and risk of disease.

### STATEMENT REGARDING FEDERALLY SPONSORED RESEARCH OR DEVELOPMENT

None.

### THE NAMES OF THE PARTIES TO A JOINT RESEARCH AGREEMENT

None.

### REFERENCE TO SEQUENCE LISTING

None.

### BACKGROUND OF THE INVENTION

Populations of circulating tumor cells can include cells able to disseminate and establish additional metastatic sites. Cancer patients with more adverse prognosis such as patients with evidence of bone metastases can be identified. However, patients without bone metastases require alternative detection methods and treatments because often CTCs have undergone epithelial-mesenchymal transition (“EMT”) and are undetectable.

A need exists therefore for alternative methods of detecting of CTCs in cancer patients.

### BRIEF SUMMARY OF THE INVENTION

Presented herein are quantitative RT-PCR tumor cell assays based on detection of expression epithelial-mesenchymal transition genes in cancer patients. The quantitative RT-PCR based assays are useful for the detection of CTCs that undergo epithelial mesenchymal transition but which cannot be detected by conventional detection methods such as FDA approved CellSearch by Veridex and AdnaTest Breast Cancer Select and AdnaTest Breast Cancer Detect by AdnaGen AG.

Specifically described herein are methods of detecting breast cancer in a human or animal, the method comprising the steps of: a) extracting CTCs from peripheral blood, plasma or serum obtained from a human or animal; b) identifying the presence of CTCs which do not have an epithelial antigen and c) further detecting the expression of at least three EMT genes using RT-PCR, wherein detection of CTCs without an epithelial antigen and the expression at least three EMT genes in the peripheral blood of patents indicates breast cancer in the human or animal. The present assay is characterized by the detection of circulating tumor cells (CTCs) undergoing epithelial mesenchymal transition (“EMT”) and is useful to detect breast cancer in a patient. By way of example, CD45-depleted (“CD45−”) peripheral blood mononuclear cells (“PBMC”) can be used to detect the expression of TWIST1, SNAIL1, SLUG, ZEB1 and FOXC2 gene transcripts and EMT by quantitative reverse transcription polymerase chain reaction (“RT-PCR”). The described assay and the associated methodology can be used in combination with conventional detection methods to identify “partial EMT” or cells which are in a partial EMT transition.

### BRIEF DESCRIPTION OF THE SEVERAL VIEWS OF THE DRAWINGS

FIG. 1 depicts the process flow for two different studies used to detect EMT associated genes.

FIGS. 2A, 2B, & 2C show the results of the spiking experiment where HMEC and TWIST1-expressing HMEC (200 cells) were spiked into 7.5 mL of peripheral blood of healthy donor. Cells were detected by different methods where results are shown in FIG. 2A, AdnaTest Select/Detect kit; FIG. 2B, CellSearch; and FIG. 2C, Relative TWIST1 expression by qRT-PCR of CD326 and CD45 depleted cells.

FIGS. 3A, 3B & 3C show the results of an experiment where SUM149 (200 cells) were spiked into 7.5 mL of peripheral blood of healthy donor. Cells were detected by different methods where results are shown in FIG. 3A, AdnaTest Select/Detect; FIG. 3B, CellSearch; and FIG. 3C, Relative TWIST1 expression by qRT-PCR of CD326- and CD45-depleted cells. Cells were detected by all methods suggesting that Twist expression may not associated with complete disappearance of EpCAM in all cells.

FIG. 4 is a depiction of a working model of CTC heterogeneity.

### DETAILED DESCRIPTION OF THE INVENTION

Recent advances in early diagnostics and treatments have drastically improved the survival rates of breast cancer patients. Nevertheless, 40,000 women die from breast cancer in the United States every year, and the majority of these deaths result from the development of metastatic disease. The formation of metastatic colonies is a continuous process, commencing early during the growth of the primary tumor as occult dissemination as documented by the early detection of cancer cells in bone marrow as disseminated tumor cells (“DTC”) and lymph nodes (“LN”) along with the detection of circulating tumor cells (“CTCs”) in the peripheral blood (“PB”). Braun et al, NEJM, 2005; Cristofanilli M, et al., Circulating Tumor Cells, Disease Progression, and Survival in Metastatic Breast Cancer, N Engl J Med. 2004 Aug. 19; 351(8):781-91.

The detection of CTCs before and during treatment represents an independent predictor of progression-free survival (“PFS”) and overall survival (“OS”) in patients with metastatic breast cancer “MBC.” Superior survival among patients with <5 CTCs was observed regardless of histology, hormone receptor and HER-2/neu status, sites of first metastases, or whether the patient had recurrent or de novo metastatic disease. Cristofanilli M, et al., Circulating Tumor Cells, Disease Progression, and Survival in Metastatic Breast Cancer, N Engl J Med. 2004 Aug 19; 351 (8):781-91; Dawood S, et al., Circulating Tumor Cells in Metastatic Breast Cancer: From Prognostic Stratification To Modification of The Staging System? Cancer 2008; 113: 2422-2430. Moreover, more recent ad-hoc analyses in patients with newly diagnosed metastatic breast cancer (“MBC”) also demonstrated that even within the subgroup of patients with bone-only metastases (typically considered having more indolent disease), there was an absolute one year survival advantage of 30.1% (p=0.0003) for the group with CTC levels <5. (De Giorgi et al, Ann Oncol, 2009). These observations were further expanded in a larger retrospective analysis including 195 patients with MBC, 103 (53%) had <5 CTCs at relapse/progression and 92 (47%) had 5 CTCs.

Patients treated with HER2-targeted therapies (15 trastuzumab and one lapatinib) and patients with inflammatory breast cancer (“IBC”) had lower CTC counts with only one case with >5 CTCs at progression during trastuzumab. Mego M, et al., Circulating Tumor Cells (CTCs) and Epithelial Mesenchymal Transition (EMT) In Breast Cancer: Describing the Heterogeneity of Microscopic Disease, Cancer Res, 2009; 69: 573s (Suppl.) (24): Abstract No. 301. Of the 92 patients with ≧5 CTCs, 83 (90%) presented with bone metastases. Of 137 patients with bone metastases at relapse/progression, 83 (61%) had ≧5 CTCs, while 54 (39%) had <5 CTCs (P=0.0122). Higher CTC numbers were detected in patients with bone metastases alone and patients with metastases in bone plus other sites relative to those with no bone metastases. Moreover, patients with ≧5 CTCs and bone metastases have higher chance to develop additional metastatic disease in distant sites compared to a) patients with bone metastases and <5 CTCs or b) patients with only visceral metastases. All those patients were receiving zoledronic acid as part of their medical treatment.

Data, therefore, suggests the CTCs population includes cells able to disseminate and establish additional metastatic sites. Moreover, we showed that by using the CellSearch technology, we can identify patients with more adverse prognosis particularly patients with evidence of bone metastases. Instead, patients with HER-2+ and mostly ER negative disease (including IBC) and without bone metastases require alternative detection methods because CTCs have undergone epithelial-mesenchymal transition (“EMT”) and are undetectable from EpCAM-based selection methods.

As a result, we propose that CTCs-targeted therapies follow a different strategy for those patients (1) with bone metastases (ER+, mostly ER− or Luminal A and/or B) identified as having ≧5 CTCs; and (2) with mostly visceral metastases and HER-2 amplified, ER/PR negative disease (HER-2, normal-like and basal-like subtypes) or rarely ER+ with CTCs by either CellSearch or EMT-detection method.

Circulating tumor cells (CTCs) are an independent predictor of survival in metastatic breast cancer (“BC”) patients. We had hypothesized that CTCs may escape detection by conventional detection methods due to epithelial-mesenchymal transition (EMT) and resultant loss of epithelial markers. As part of our initial studies, we had aimed to detect CTCs based on expression of EMT genes in peripheral blood of BC patients. 31 patients (15 primary BC and 16 metastatic BC) and 20 healthy donors (HD) were initially analyzed. Isolated peripheral blood mononuclear cells (PBMC) were depleted of cells of hematopoietic origin (CD45+) by anti-CD45 coated magnetic beads. CD45-depleted (CD45−) PBMC were interrogated for expression of TWIST1, SNAIL1, SLUG, ZEB1 and FOXC2 gene transcripts by quantitative reverse transcription polymerase chain reaction (“RT-PCR” or “RTPCR”).

Expressions of gene transcripts in CD45− PBMC from patients were compared to those of CD45− PBMC of healthy donors (“HD”). Concurrently, a peripheral blood sample was collected for determination of CTC by CellSearch. Eighteen patients had detectable CTC by CellSearch; Median CTC count was 2 (range; 0-750) per 7.5 mL of peripheral blood. Expression above 2 SD (standard deviation) from the mean expression in healthy donors of TWIST1, SNAIL1, SLUG, ZEB1 and FOXC2 was detected in CD45− PBMC in 6%, 6%, 6%, 10% and 3% of patients, respectively. Seven (23%) patients had overexpressed at least one of these genes.

Expression of SLUG was detected in patient CD45− PBMC samples, and not in those of HD. There was no correlation between CTC count and EMT genes expression. Moreover, in additional studies we spiked TWIST1 transformed human mammary epithelial cells (“HMEC”) with EMT phenotype and vector HMEC into peripheral blood of HD. Cells with EMT phenotype were not detected by conventional methods (CellSearch, Veridex LLP, Raritan, N.J.; AdnaTest Breast Cancer Select and AdnaTest Breast Cancer Detect, AdnaGen AG, Langenhagen, Germany), but were detected based on expression of TWIST1 in CD45-depleted PBMC. We suggest that EMT genes are involved in dissemination of CTCs. Loss of epithelial antigen on CTC due to EMT, triggered by high expression of these genes, may be responsible for their undetection by current CTC detection methods and this development may lead to recurrence of disease.

The detection EMT in cancer patients using RT-PCR is a novel diagnostic method and a prognostic tool not only for breast cancer patients but other tumors of epithelial origin. Our method is more sensitive and able to detect CTCs, after EMT which is not detected using convention methods of detection. In addition, EMT genes are associated with stem cell phenotype and therefore the prognostic value of our assay should be more powerful compared to assays using epithelial markers for CTC detection.

In addition, we analyzed aphaeresis samples from 14 breast cancer patients treated by autologous stem cell transplantation. Median CTC measured by CellSearch was 0. In a median follow up of 12 months, 3 patients of the 14 patients experienced disease recurrence. Using our approach as described herein, we were able to discriminate patients with recurrent disease based on expression of TWIST1 gene in aphaeresis samples at the time of transplantation. Moreover, we spiked TWIST1 transformed human mammary epithelial cells (HMEC) with EMT phenotype and vector HMEC (control) into peripheral blood of healthy donors. Cells with EMT phenotype were not detected by conventional methods (CellSearch by Veridex; AdnaTest Breast Cancer Select and AdnaTest Breast Cancer Detect by AdnaGen AG), but were detected based on expression of TWIST1 in CD45-depleted PBMC.

This suggested that EMT genes are involved in dissemination of CTCs. Loss of epithelial antigen on CTC due to EMT, triggered by high expression of these genes, is believed responsible for their un-detection by current CTC detection method and this development may lead to recurrence of disease. Hence, the method of detection of EMT in cancer patients using RT-PCR and as taught herein is a novel diagnostic method and a prognostic tool not only for breast cancer patients but other tumors of epithelial origin.

CTCs-targeted therapy in MBC with bone metastasis Metastatic colonization involves reciprocal interactions between tumor cells and a foreign microenvironment. Microenvironments consist of extracellular matrix and normal cells such as fibroblasts, endothelial cells and infiltrating inflammatory cells. Products of these resident and transient cells include growth factors, chemokines, cytokines and proteases. In breast cancer the issue of site-specific metastasis has provided the opportunity to investigate the potential role of chemokines, particularly with regards to CXCR4 and RANKL. RANKL (also referred to as OPGL, TRANCE or ODF) is a member of the tumour necrosis factor (“TNF”) family of cytokines that binds to its receptor RANK to control osteoclast differentiation, activation and survival. Osteoprotegerin OPG is a soluble decoy receptor for RANKL that blocks ligand binding to RANK, thereby preventing the signaling required for osteoclast differentiation and activation. RANK is also constitutively expressed in normal mammary gland epithelial cells, but RANKL expression is induced by sex hormones during pregnancy.

Genetically, both RANKL and RANK are essential for the development of the lactating mammary gland during pregnancy and for lymph node organogenesis in mouse embryos. Furthermore, RANK has shown expressed on many different epithelial tissues and epithelial tumour cells, and can activate specific downstream signaling pathways. Interestingly, RANKL also stimulates migration of primary breast epithelial cells and osteoclasts, establishing that RANKL-induced cell migration also occurs in normal, non-transformed cells. Importantly, inhibition of RANKL/RANK signaling by OPG in vivo markedly and selectively reduces bone metastasis and tumour burden in a melanoma model that does not activate osteoclasts.

New molecular therapeutics designed to inhibit bone metastasis have entered clinical testing. Among them, the humanized monoclonal antibody denosumab (AMG 162) binds RANKL and inhibits its action. Recently, a study evaluated denosumab in a randomized double-blind setting to determine the safety and efficacy of this antibody in patients with breast cancer or multiple myeloma with confirmed bone metastasis. Patients received a single dose of either denosumab or pamidronate, and bone anti resorptive effect was assessed by changes in urinary and serum N-telopeptide levels. Interestingly, denosumab treatment resulted in a decrease in levels of urinary and serum N-telopeptide that lasted for 84 days and significant decrease in the bone-events.

We developed a single-arm pilot study including 42 patients with HER-2 negative MBC with bone-disease and ≧5 CTCs about to start a new line of chemotherapy. Those patients usually receive chemotherapy treatment and bisphosphonates (zoledronic acid) as standard of care. After the first cycle of standard chemotherapy, at 3-4 week, patients will be re-evaluated for CTCs detection. If they have still persistent ≧5 CTCs they will continue their chemotherapy but zoledronic acid will be discontinued. Patients will then be receiving treatment with chemotherapy+Denosumab at 120 mg SQ every 4 weeks×2 doses. Patients will have repeated measurement of CTCs and be re-evaluated by standard imaging (CT of metastatic sites of PET/CT) as for their primary team at 8-9 weeks from time of treatment with denosumab. At that time we will determine that fraction of patients with normalization of CTCs, defined as CTC<5. Patients with normalization of CTCs value and no evidence of progression or response will continue the same treatment (chemotherapy with denosumab).

CTCs-targeted therapy in EMT-enriched breast cancer. Despite recent advances in our understanding of the signals/processes that drive primary breast tumor formation and growth, the molecular characteristics of CTCs and the mechanisms underlying their generation remain poorly understood. The currently available CellSearch™ CTC Test (Veridex Corporation, Warren, N.J.), used to enumerate CTCs in the whole blood of cancer patients, is the only FDA-cleared diagnostic test for CTC detection and enumeration. Following enrichment using magnetic beads coated with EpCAM-specific antibodies, isolated cells are stained for epithelial (cytokeratins 8/18/19) and leukocyte (CD45) antigens and their nuclei counterstained with DAPI, to enable subsequent quantification by fluorescence microscopy. Thus, using this methodology, CTCs are currently isolated as nucleated cells lacking CD45 expression that express epithelial antigens, e.g. EpCAM, cytokeratin or MUC-1. However, recent clinical and laboratory studies found that EpCAM-enrichment methods could rarely detect CTCs in patients with HER-2 amplified disease or inflammatory breast cancer (IBC). Moreover, the detection of CTCs, by the CellSearch™ system, had no prognostic value in HER-2+ patients receiving trastuzumab. A possible explanation for these results is that in some cases CTCs may undergo partial or complete EMT prior to entering the circulation. Indeed, reactivation of the EMT program has been shown to facilitate invasion and metastasis in many types of breast cancers including HER-2-amplified Thiery et al, Cell 2009.

During EMT, epithelial cells lose cell-cell contacts and cell polarity, lose epithelial gene expression, acquire mesenchymal gene expression, and undergo major changes in their cytoskeleton, and this enables them to acquire a mesenchymal appearance with increased motility and invasiveness. The development of tumor metastasis, which is often enabled by EMT, is associated with disseminated cancer cells that would demonstrate a self-renewal capability, similar to that exhibited by stem cells, in order to spawn macroscopic metastases. Thiery et al, Nat Rev Mol Cell Bio, 2006. The EMT process enables cancer cell dissemination and may also endow disseminating cancer cells with a self-renewal capability. A link exists between less-differentiated stem cells and the mesenchymal-appearing cells generated by EMT. Mani et al, Cell 2008.

For example, a CD44+ stem cell signature has been demonstrated in primary invasive tumors associated with a higher risk of distant metastasis. Park S Y, Clin Cancer Res 2010. In the same patients the distant metastases were enriched for more luminal epithelial CD24+ cells, implying a phenotypic switch during tumor progression or clonal selection for cells with CD24+ phenotype. Bloushtain-Qimron et al, Proc Natl Acad Sci USA 2008. Furthermore, there is evidence that breast residual disease after neoadjuvant therapy is enriched in CSCs (CDd44+/CD24−) suggesting clones resistant to standard therapies. Creighton et al, Proc Natl Acad Sci USA, 2009.

Hence, we conclude that EMT generates cancer stem cells that are circulating in peripheral blood and are responsible for progression of disease and, in establish distant metastases, there is a reversion of this process to mesenchymal-to-epithelial transition. Our effective therapeutic strategy includes, therefore, a combination of standard systemic therapies, e.g. chemotherapy or endocrine therapy (epithelial metastasis) and novel use of new and existing molecules to modulate EMT (mesenchimal/stem cell process).

Several biomarkers have been associated with the EMT phenotype including expression levels of established EMT inducers (e.g. Twist, Snail, Slug, FOXC1, FOXC2, ZEB1, and ZEB2) Akt2 and PI3Kα and their expression as been used to detect cancer stem cells in tissue or peripheral blood. Akt and PI3K-inhibitors or other therapies may be able to induce reprogramming and differentiation of such cancer cells.

Understanding the molecular bases of gene silencing and epigenetic changes facilitates the use of our methods. For example, gene silencing is regulated by the opposing actions of histone acetyltransferases (“HTAs”) and histone deacetylases (“HDACs”). HTA activity removes acetyl moieties causing relaxation of chromatin and permitting various transcription factors to interact with DNA. Thiagalingam S, et al. Ann NY Acad Sci, 2003. In contrast, HDAC activity condenses chromatin, preventing access to transcriptional factors and leading to transcriptional repression. HAT inactivity and HDAC overactivity have been associated with tumorigenesis. Marks PA et al. Adv Cancer Res, 2009. Overexpression of HDAC1/HDAC2 has been associated with EMT (including Snail overexpression) in highly metastatic pancreatic cancer models. von Burstin J et al, Gastroenterology 2009.

Similarly, Snail repression of E-cadherin expression (epithelial marker) has been related to overexpression of HDAC1/HDAC2. Peinado H et al. Mol Cell Biol 2004. Moreover, CSCs (CD44+) have shown been hypomethylated (including FOXC1) compared to cells with more differentiated phenotype in human breast cancer. Bloushtain-Qimron et al, Proc Natl Acad Sci, 2008.

HDAC-inhibitors are representative of a novel class of agents having the capability to affect CSCs. Multiple preclinical studies have demonstrated that HDACs can promote differentiation and apoptosis by regulating histone and nonhistone protein expression including heat shock protein (hsp) 90, estrogen (ER) α, HER-2 and p53. Bali P et al, J Biol Chem, 2005; Yang X et al, Cancer Res 2001; Weng S, et al. J Clin Oncol 2009. In particular, HDAC-inhibition demonstrated able to restore expression of ERα in ER-negative cell lines. Yang X et al, Cancer Res 2001. Furthermore, vorinostat proved effective in increasing efficacy of chemotherapy (carboplatin and paclitaxel) in NSCLC indicating increasing apoptotic effect. Owonikoko et al, Int J Cancer 2010; Ramalingam et al, J Clin Oncol 2010.

Hence, the novel methodology described herein allows detection of cancer cells with either epithelial or mesenchymal phenotype in the peripheral blood. A component of these cells has undergone partial or total EMT, a characteristic of CSCs. Using HDAC-inhibitors in combination with chemotherapy can result in increased efficacy of therapy on established metastases (synergistic, proapoptotic effect); decreased CTCs, including both EpCAM+ and/or EMT+ with increased differentiation (re-expression of E-cadherin, ERα, decrease vimentin); and prolongation of treatment benefit by increased PFS (historical control) and reduction of metastatic seed (additional mets after initial site).

A number of studies have demonstrated that the presence of tumors cells within the circulation (circulating tumor cells; “CTCs”) is an independent prognostic factor of progression-free survival (“PFS”) and overall survival (“OS”) in patients with metastatic breast, colon and prostate cancers, respectively. Cristofanilli M, et al., Circulating Tumor Cells, Disease Progression, and Survival in Metastatic Breast Cancer, N Engl J Med. 2004 Aug 19;351(8):781-91; Cohen S J, et al., Relationship of Circulating Tumor Cells To Tumor Response, Progression-Free Survival, and Overall Survival In Patients With Metastatic Colorectal Cancer, J Clin Oncol. 2008; 26: 3213-3221; de Bono J S, et al., Circulating Tumor Cells Predict Survival Benefit From Treatment In Metastatic Castration-Resistant Prostate Cancer, Clin Cancer Res. 2008; 14: 6302-6309. Commercially-available CTC detection kits including the FDA-cleared CellSearch™ system (Veridex Corporation, Warren, N.J.) and the semi-quantitative qRT-PCR based AdnaTest (Breast Cancer Select/Detect kit or AdnaTest AdnaGen AG, Langenhagen, Germany), exploit the expression of the epithelial cell marker CD326 (aka ESA or Ep-CAM) and cytokeratins (CK) by CTCs Allard et al., 2004; Cristofanilli et al., 2004, supra; Ignatiadis M, et al., Prognostic Value Of The Molecular Detection of Circulating Tumor Cells Using A Multimarker Reverse Transcription-PCR Assay For Cytokeratin 19, Mammaglobin A, and HER2 in Early Breast Cancer, Clin Cancer Res. 2008; 14: 2593-2600; Pachmann K, et al., Monitoring The Response of Circulating Epithelial Tumor Cells To Adjuvant Chemotherapy In Breast Cancer Allows Detection of Patients At Risk of Early Relapse, J Clin Oncol. 2008; 26: 1208-1215. On the other hand, the AdnaTest EMT-1/Stem Cell Select/Detect kit (AdnaTest-EMT) is available for the detection of the CTCs with expression of EMT associated genes after EpCAM enrichment. Aktas B, et al., Stem Cell and Epithelial-Mesenchymal Transition Markers Are Frequently Overexpressed in Circulating Tumor Cells of Metastatic Breast Cancer Patients, Breast Cancer Res. 2009; 11: R46.

During the metastatic progression of carcinoma, cancer cells detach from the primary tumor, traverse the peripheral circulation, reach distant sites and potentially establish secondary tumors. A number of studies suggest that carcinoma cells often activate a transdifferentiation program termed the epithelial mesenchymal transition (EMT) to acquire the ability to execute the multiple necessary steps of this invasion—metastasis cascade. Fidler I J., The Pathogenesis of Cancer Metastasis: The ‘Seed and Soil’ Hypothesis Revisited, Nat Rev Cancer. 2003; 3: 453-458. During an EMT, epithelial cells lose cell-cell contacts and cell polarity, down regulate epithelial markers, acquire mesenchymal gene expression, and undergo major changes in their cytoskeleton that enables them to acquire a mesenchymal appearance with increased motility and invasiveness.

Epithelial can be induced by several alternative signaling pathways, notably those involving the cooperation between TGF-β1 signaling with oncogenic Ras or receptor tyrosine kinases, Wnt, Notch, and the signaling activated by Hedgehog (Moustakas, 2007). In addition, certain developmental transcription factors, specifically Snail1, Slug, Zeb1, E12/E47, Goosecoid, FOXC2 and Twist, can promote this transition. The expression of some of these transcription factors has been found to be induced during tumor progression and is associated with resistance to apoptosis. Yang, J., Weinberg R A., Epithelial-Mesenchymal Transition: At the Crossroads of Development and Tumor Metastasis, Developmental Cell 2008; 14: 818-829; De Wever, O, et al., Molecular and Pathological Signatures of Epithelial-Mesenchymal Transitions at The Cancer Invasion Front, Histochem Cell Biol 2008; 130: 481-494.

Previously, we demonstrated that EMT and EMT-inducing transcription factors play a key role during breast cancer progression by facilitating the invasion and dissemination of cancer cells. Yang J, Mani S A, et al., Twist a Master Regulator of Morphogenesis, Plays an Essential Role in Tumor Metastasis, Cell. 2004; 117: 927-939; Mani S A, Yang J et al., Mesenchymal Forkhead 1 (FOXC2) Plays a Key Role in Metastasis and is Associated With Aggressive Basal-like Breast Cancers, Proc Natl Acad Sci USA. 2007; 104: 10069-10074; Mani S A, et al., The Epithelial-Mesenchymal Transition Generates Cells With Properties of Stem Cells, Cell. 2008 May 16; 133(4):704-15. In addition, we found that blocking the expression of Twist using shRNA in the highly metastatic 4T1 cells led to a reduction in the number of CTCs in mice bear metastatic xenograft mammary tumors, using exogenous puromycin resistance to identify circulating cells. Yang et al., Cell 2004, supra. Hence, the expression of epithelial-associated surface markers, such as EpCam, may not be the optimal technique for identifying CTCs, since it is likely that some CTCs in breast cancer patients have a more mesenchymal phenotype.

In addition, the induction of an EMT in immortalized human mammary epithelial cells (HMECs) results the expression of stem cell markers and acquisition of functional cancer stem cell properties. This finding illustrates a direct link between the EMT phenotype and cancer stem cell properties (Mani, 2008) and suggests that EMT may be at least responsible for the heterogeneity of some breast tumors.

Since EMT is known to play a pivotal role during cancer progression (Yang et al., 2004; Mani et al., 2007), we hypothesized that current CTC detection kits do not detect CTCs that have undergone EMT. The aim of our study below was to determine the feasibility of detecting CTCs based on the mRNA expression of EMT-regulating transcription factors in the PB of patients with early stage breast cancer and correlate these findings with the number of CTCs detected by conventional methods and tumor characteristics.

In a prospective pilot translational study, we detected EMT genes overexpression in 17.3% of patients with breast cancer compared to normal donors. SLUG was most commonly overexpressed, while SNAIL1 and ZEB1 were not overexpressed in any of the samples. Moreover, TWIST1 expression was more often detected in patient samples compared to healthy donor.

EMT increases cell motility and seems to play an important role in intravasation and release of CTCs. It has been shown that the expression of genes involved in EMT in breast cancer is associated with poor prognosis. Martin T A, et al., Expression of the Transcription Factors Snail, Slug, and Twist and Their Clinical Significance in Human Breast Cancer, Ann Surg Oncol. 2005; 12: 488-496. In addition, we have linked EMT with cancer stem cell properties, which have independently been linked to increased therapeutic resistance. Mani S A, et al., The Epithelial-Mesenchymal Transition Generates Cells With Properties of Stem Cells, Cell. 2008 May 16;133(4):704-15; Hollier et al, 2009, Li, X, et al., Intrinsic Resistance of Tumorigenic Breast Cancer Cells To Chemotherapy, J Natl Cancer Inst. 2008; 100: 672-679; Woodward et al, 2007.

Interestingly, we observed that patients after neoadjuvant chemotherapy have significantly higher overexpression of EMT genes, compared to untreated patients (41.2% vs. 10.34%; p=0.007), suggesting a possible chemotherapeutic resistance of EMT-derived CTCs. Moreover, we recently reported an increase in disseminated tumor cells with cancer stem cell phenotype (defined as ALDH+/CD45−/CD326+CD44+CD24−) in bone marrow of primary breast cancer patients following neoadjuvant chemotherapy. Reuben J M, et al., Disseminated Tumor Cells in Primary Breast Cancer: Evaluation of The Percentage of Breast Cancer Stem Cells in Bone Marrow Aspirates of Patients Receiving Neoadjuvant Chemotherapy, J Clin Oncol 27:15s, 2009 (suppl; abstr 505).

The expression in EMT genes also correlated with pCR after chemotherapy leading us to suggest that EMT genes overexpression may be a prognostic marker for breast cancer. Pathologic complete remission is a surrogate marker for cure in early breast cancer after neoadjuvant chemotherapy; however, the detection of EMT genes could represent a novel blood-based surrogate marker for failure to achieve pCR. Harris L N, et al., Preoperative Therapy for Operable Breast Cancer, In: Harris J R, et al. Diseases of the Breast. 3^{rd} ed. Philadelphia, Pa.: Lippincott Williams & Wilkins, 2004: 929-94. Nevertheless, a longer follow-up period is needed to determine the prognostic value of overexpression of EMT genes in PB relative to clinical outcome.

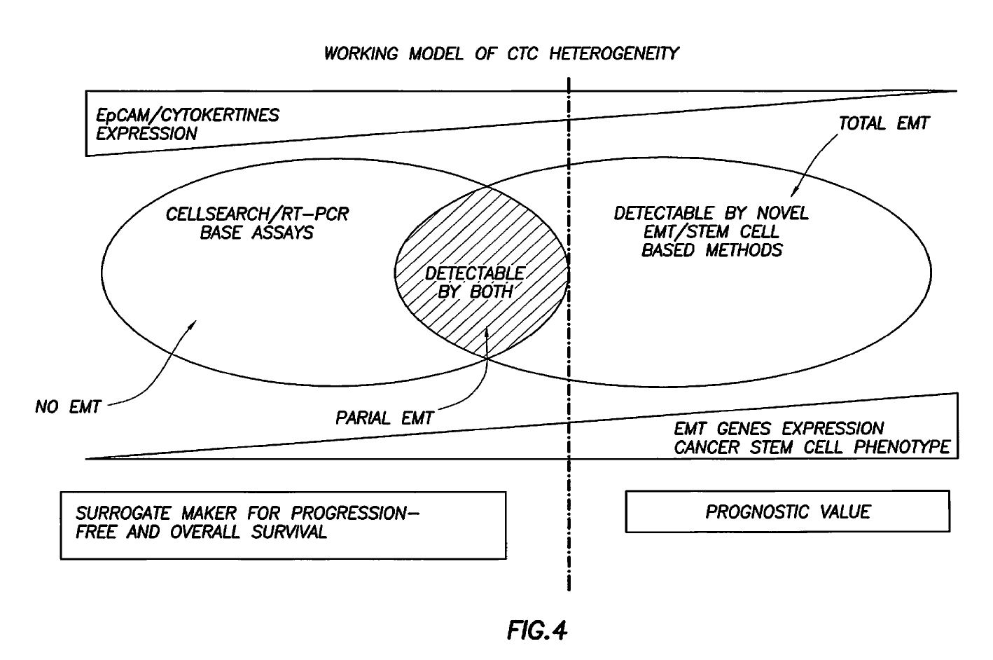

We found a weak association between CTC positivity measured by either CellSearch™ or AdnaTest and EMT genes overexpression, which suggests that these tests are able to detect cells with partial EMT. Recently, Aktas et al showed that more than 60% of CTCs detected by AdnaTest have expression of genes associated with EMT and stem cell phenotype (TWIST1, PI3K, Akt, and ALDH). Taken together, the data can support the concept that there is a continuum of development of CTCs that range from one end of the spectrum (epithelial phenotype) to the other end of the spectrum (mesenchymal phenotype) and including those with a partial EMT phenotype. The CTCs with partial EMT phenotype are capable of co-expressing both epithelial and mesenchymal antigens (FIG. 4). As shown earlier, HMEC-TWIST1 cells were not detected using either AdnaTest or CellSearch™ systems, suggesting that CTC with complete EMT phenotype are not detected by the EpCAM-based detection tests. However, CTCs with complete EMT phenotype may represent CTCs that are most resistant to therapy and exhibit cancer stem cell properties.

The prognostic value of CTCs measured by CellSearch™ is well established; however, it not clear if this prognostic value is due to detection of CTCs with partial EMT features (and CSC phenotype) or simply to CTCs with epithelial characteristics. As previously described by Allard et al., apoptotic enucleated cells could be observed but are not counted as CTCs by CellSearch™. Allard W J, et al., Tumor Cells Circulate In The Peripheral Blood of All Major Carcinomas But Not In Healthy Subjects or Patients With Nonmalignant Diseases, Clin Cancer Res 2004;10: 6897-6904. Based on this, the lack of correlation between tumor burdens (measured by Sweerton score), tumor markers and CTCs support the concept that the majority of CTCs enumerated by CellSearch™ have partial EMT phenotype.

HER2/neu amplified and IBC are most aggressive forms of breast cancer. Therefore, we suggest that the CTC count will mirror disease biology. Surprisingly, metastatic breast cancer patients with HER2 amplified disease do not have higher prevalence of ≧5 CTCs/7.5 mL of PB and metastatic IBC patients have CTC counts even lower than that of non-IBC patients. Cristofanilli, 2004, supra.; Mego M, et al., Circulating Tumour Cells in Metastatic Inflammatory Breast Cancer. Annals of Oncology, 2009, 20: 1824-1828; Dawood S, et al., Circulating Tumor Cells in Metastatic Breast Cancer: From Prognostic Stratification To Modification of The Staging System? Cancer 2008; 113: 2422-2430. HER2-amplified breast cancer subtypes with up regulated EMT inducing pathways and total EMT appear to lead to down regulation of epithelial markers that impair CTCs detection. EMT might be one of the mechanisms that regulate the metastatic ability of IBC. In an experimental model, it has been shown that HER2 induces EMT. Mani, 2008, supra.; Kleer C, et al., Molecular Biology of Breast Cancer Metastasis. Inflammatory Breast Cancer: Clinical Syndrome and Molecular Determinants, Breast Cancer Res 2000; 2: 423-42. In the present study, we did not observe high over-expression of EMT genes in tumor with poor prognosis features (high grade, HER2/Neu amplification, hormone receptor negative tumors); however, this may be due to sample size as well as under representation of this subgroup in the study population.

In conclusion, we found that the cancer cells with complete EMT phenotype cannot be detected using conventional CTC detection methods such as CellSearch™ or AdnaTest. However, cancer cells with complete EMT phenotype can be detected in PB depleted of CD45⁺ cells using a simple qRT-PCR based method. Since identification of cells with EMT phenotype and/or stem cell-like properties increases the biological significance of these cells in the progression of epithelial cancers, we suggest that detection of CTC using this modified method could add new prognostic information in a broad range of epithelial tumors and could potentially lead to identification of novel therapeutic targets. As such, the assay as presented herein is direct to detecting CTC based on overexpression of EMT genes in peripheral blood. We showed that after EMT, CTCs are present in peripheral blood of breast cancer patients. Such cells are enriched in patients after neoadjuvant chemotherapy and in patients without pathologic complete response to therapy. Loss of epithelial antigen on CTCs due to EMT, triggered by high expression of EMT-associated genes, may be responsible for their undetection by conventional methods. Detection and further phenotypical characterization of these cells could provide new prognostic information and could lead to identification of novel therapeutic targets.

### EXAMPLE I

### Circulating Tumor Cells (CTCs) and Epithelial Mesenchymal Transition (EMT) in Breast Cancer: The Heterogeneity of Microscopic Disease

Tumor cells are hypothesized to undergo epithelial mesenchymal transition (EMT) prior to entering the circulation. This passage through EMT can result in the loss of epithelial markers, which could cause these cells to escape detection by conventional methods for detecting cancer cells within the peripheral blood, or circulating tumor cells (CTCS). This study reports the detection of CTCs based on the expression of EMT genes in the peripheral blood (PB) of breast cancer (BC) patients. Our study included 75 BC patients in stages I-IV and 30 healthy donors. Seventeen patients received neoadjuvant chemotherapy (NACT). Peripheral blood mononuclear cells were isolated from 5 mL of PB depleted of hematopoetic and/or of EpCAM positive cells. The CD45-depleted cells were interrogated for the expression of EMT-related genes by quantitative RT-PCR. Concurrently, PB sample was analyzed by CellSearch™ and/or AdnaTest assays. In total, 17.3% of patients overexpressed at least one of the EMT-related genes. There was a weak correlation between expression of EMT-related genes and detection of CTCs by CellSearch™ and/or AdnaTest. Patients after NACT had higher expression of EMT genes compared to patients without NACT (41.18% vs. 10.34%; p=0.007). Based on the results obtained, we suggest that CTCs with an EMT phenotype are present in BC patients' PB. The loss of EpCAM on CTCs due to EMT may result in the underestimation of CTCs by current conventional methods thus current methods may not fully account for the heterogeneity of breast cancer cells.

### Patients and Methods

As a part of two translational studies at the University of Texas M.D. Anderson Cancer Center, from November 2008 to May 2009 a total of 75 breast cancer patients with stage I-IV were included. The first study consists of 52 patients (study A) was aimed to determine the prognostic value of CTC measured by CellSearch™ system and the AdnaTest. The second study (Study B) consists of 23 patients with inflammatory breast cancer (IBC) and/or metastatic breast cancer aimed to determine immune status in IBC patients. For every patient a complete diagnostic evaluation to exclude the presence of distant metastasis was performed. Patients with concurrent malignancy other than non-melanoma skin cancer in previous 5 years were excluded. Patient demographic data included age, tumor histology, hormone receptor status, HER2 status, and systemic therapy and correlation with expression of EMT-related genes in PB. We enrolled both newly diagnosed patients as well as patients after completion of neoadjuvant therapy (NACT).

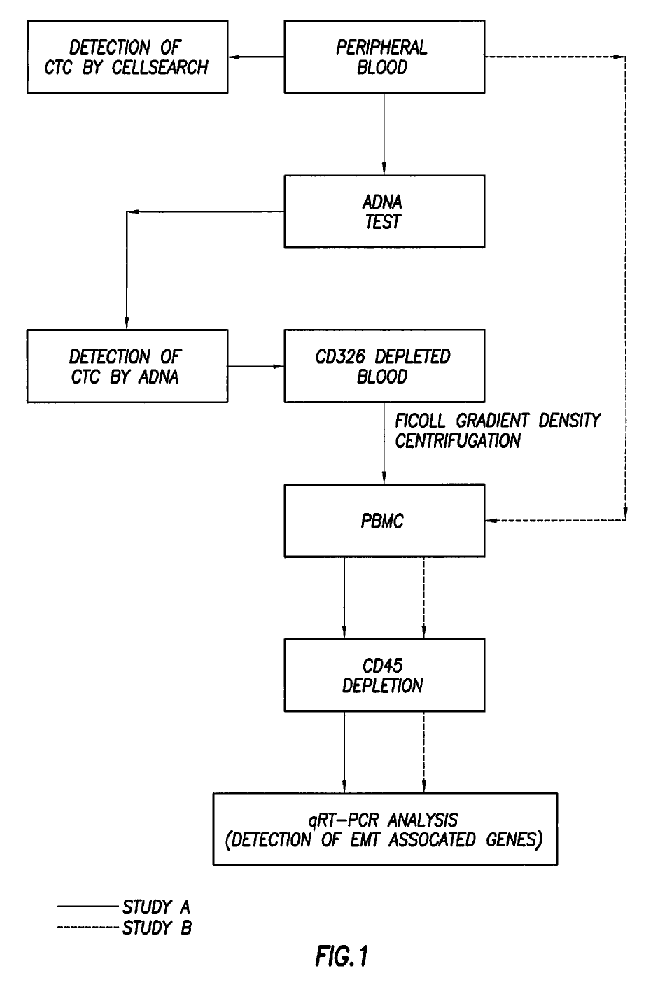

Healthy donors were 30 healthy women blood donors (HD) who consented and were recruited. The study was approved by the Institutional Review Board. All samples were processed as described in FIG. 1.

### Detection of CTC in Peripheral Blood Using CellSearch™ System

The CellSearch™ system (Veridex Corporation, Warren, N.J., USA) was used to detect CTC in 7.5 mL of whole PB as previously described (Allard, 2004). Briefly, PB samples were subjected to enrichment of EpCAM+ cells with anti-EpCAM coated ferrous particles. CTCs were defined as nucleated cells lacking surface expression of CD45 but expressing cytoplasmic CK 8, 18, or 19. Cristofanilli, 2004, supra. Samples were considered to be positive if they had ≧1 CTC per 7.5 mL PB.

### Detection of CTC Using AdnaTest Breast Cancer Select and AdnaTest Breast Cancer Detect

Peripheral blood (5 mL) was collected in AdnaCollect tubes (AdnaGen AG, Langenhagen, Germany) and stored at 4° C. and processed within 4 hours of collection, according to the manufacturer's instructions (AdnaGen). Briefly, PB samples were enriched for epithelial cells with EpCAM coated magnetic beads. Thereafter, RNA was isolated from EpCAM-enriched cells, followed by reverse transcription to cDNA and PCR. The PCR product was interrogated for expression of tumor associated antigens (EpCAM, MUC1, and HER2) and housekeeping gene, β-actin. The sample was considered to be CTC positive, if the PCR product expressed at least one of the tumor associated antigens.

### Detection of CTC by Quantitative RT-PCR Based on Detection of EMT Associated Genes

In study A, AdnaTest Select Breast Cancer assay was used to deplete peripheral blood of EpCAM+ cells and the residual cells subjected to ficoll-hypaque density gradient to isolate EpCAM-depleted peripheral blood mononuclear cells (PBMCs). In study B, PBMCs were isolated without previous depletion of CD326 expressing cells. The PBMCs were collected, washed twice with sterile phosphate buffered saline (PBS) and the isolated PBMCs (up to 1×10⁷) were incubated with 40 μL of magnetic beads coated with anti-CD45 antibody (Miltenyi-Biotec, Auburn, CA) for 15 minutes on 4° C. Thereafter, the cells were passed through a magnetic-filled column on an AutoMACSPro Cell Separator (Miltenyi-Biotec, Auburn, Calif.) using the negative selection protocol (DEPLETE protocol) to enrich for CD45-depleted CTCs with a possible EMT phenotype. The CD45-depleted sample was recycled through the magnet-filled column to deplete the sample of any residual CD45⁺ cells using the MACS DEPLETES protocol. Using this approach the median depletion of CD45 expressing cells was 97.4% (range 90.1%-99.8%).

RNA Extraction and cDNA Synthesis

CD45-depleted cells were mixed with Trizol LS Reagent (Invitrogen) and stored at −80° C. until it was necessary to extract RNA according to manufacturer's instructions. The isolated RNA was dissolved in diethylpyrocarbonate-treated water, treated by DNAse (Ambion INC, Austin, Tex.) to minimize contamination by genomic DNA and stored at −80° C.

All RNA preparation and handling steps took place in a laminar flow hood, under RNase free conditions. RNA concentration was determined by absorbance readings at 260 nm. RNA extracted from human mammary epithelial cells transformed by TWIST1 and SUM 149 cell line was used as positive control. Reverse transcription of RNA was carried out with cDNA archive kit (ABI, Foster City, Calif.). cDNA was synthesized from the total RNA isolated from CD45-depleted PBMC of breast cancer patients and healthy volunteers according to manufacturer's instructions.

### Identification of Gene Transcripts in Unselected and CD45⁻ Enriched Subsets

Synthesized cDNA was subjected to RT-PCR to detect EMT-related gene transcripts (TWIST, SNAIL1, SLUG, ZEB1 and FOXC2) and EpCAM. In brief, 2.5 μL of cDNA was placed in 25 μL of reaction volume containing 12.5 μL, of TaqMan Universal PCR Master Mix, No AmpErase UNG, 8.75 μL water and 1.25 μL of primers. The primers, TWIST1: Hs00361186_m1, SNAIL1: Hs00195591_m1, SLUG: Hs00161904_m1, ZEB1: Hs01566408_m1, FOXC2: Hs00270951_s1 and EpCAM: Hs00158980_m1, were purchased from Applied Biosystems, (ABI, Foster City, Calif.). Primers were designed to span exon-exon boundaries with the exception of FOXC2, due to lack of availability.

Amplification was performed in an ABI Fast 7500 Real Time PCR system (Applied Biosciences) using the cycling program: 95° C. for 10 min; 40 cycles of 95° C. for 15 s, 60° C. for 60 s. All samples were analyzed in triplicate. Quantification of target genes and an internal reference gene was performed using a fluorescence based RT-PCR. Calibrator samples were run with every plate to ensure consistency of PCR. DNA contamination was assessed by performing PCR on the non-reverse transcribed portion of each sample. For all fluorescence based qRT-PCR, fluorescence was detected after 0-40 cycles for the control and marker genes in single-plex reactions, which allow for the deduction of the cycles at threshold (CT) value for each product. The CT value is a PCR cycle at which a significant increase in fluorescence is detected due to exponential accumulation of PCR products. Expression of the genes of interest was calibrated against expression of the housekeeping gene, GAPDH. Target cDNA was quantified using the delta-CT method with the formula: ½̂^{Ct (target-GAPDH)}. For a combined marker a test was considered to be positive if any of the markers was positive.

### Cell Culture

The SUM 149 IBC cell line used for the study has been developed from pleural effusions of breast cancer patients (Ethier, 1993). SUM 149 cells were suspend in F-12 Hams medium (Gibco™, CA) supplemented with 5% fetal bovine serum (Tissue Culture Biologicals, Seal Beach, Calif.), 5 μg/mL of insulin, and 1 μg/mL hydrocortisone and cultured in a humidified incubator at 37° C. with 5% CO₂. The immortalized HMEC and HMEC transformed by TWIST1 were maintained as previously described (Elenbaas et al., 2001). TWIST1-HMEC was derived as previously described (Mani et al. 2008).

### Statistical Considerations

Patient characteristics were summarized using the median (range) for continuous variables and frequency (percentage) for categorical variables. Fisher's Exact test was used to assess the association between EMT gene over-expression and other patient characteristics. A p-value of <0.05 was deemed to be of statistical significance. All statistical analyses were conducted using SAS 9.1 (SAS Institute Inc., Cary, N.C.).

Results Detection of Spiked Cells with EMT Phenotype in Peripheral Blood of Healthy Donors

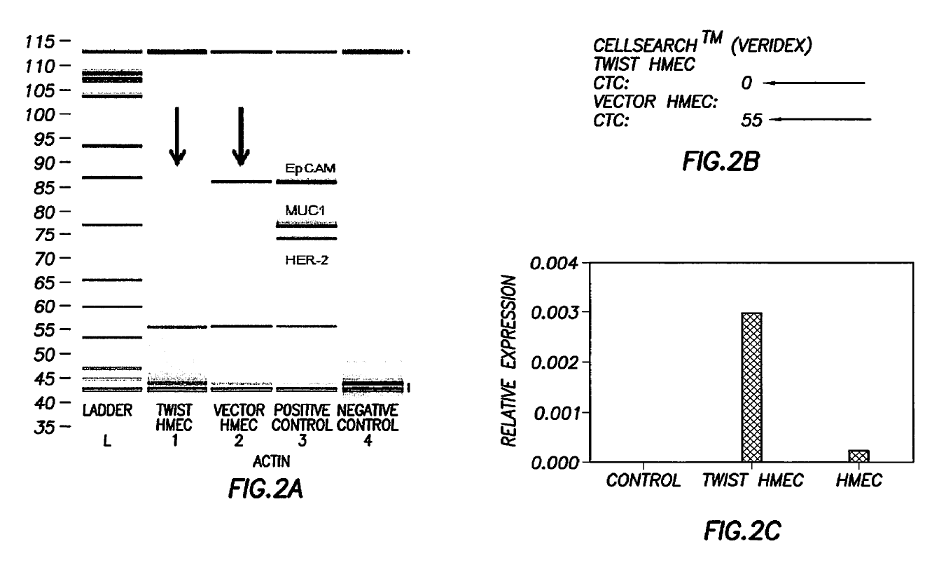

To test first our hypothesis, we used HMECs that have undergone EMT by the ectopic expression of TWIST1 (HMEC-TWIST) and the respective control vector infected HMECs (HMEC-Control). In addition, we employed an inflammatory breast cancer cell line, SUM 149, that had undergone partial EMT. To test whether the HMECs that had undergone EMT could be detected by the available assays, we spiked 7.5 mL and 5.0 mL of healthy donor PB with either 50-1000 HMEC-TWIST or 50-1000 SUM 149 cells. These samples were then analyzed for CTCs using CellSearch™ (Allard et al., 2004) and AdnaTest (AdnaGen AG) as well as for cells with an EMT phenotype using the AdnaTest EMT kit. Aktas B, et al., Stem Cell and Epithelial-Mesenchymal Transition Markers are Frequently Overexpressed in Circulating Tumor Cells of Metastatic Breast Cancer Patients, Breast Cancer Res. 2009; 11: R46. As shown in FIG. 2, while TWIST1 HMEC was not detected by CellSearch and AdnaTest due to complete EMT compared to untransformed HMEC with epithelial phenotype, TWIST1-expressing HMECs were detected based on TWIST1 expression in CD326- and CD45-depleted fraction of peripheral blood. The highest TWIST1 expression in CD45-depleted peripheral blood of healthy donors was 2×10⁻⁵.

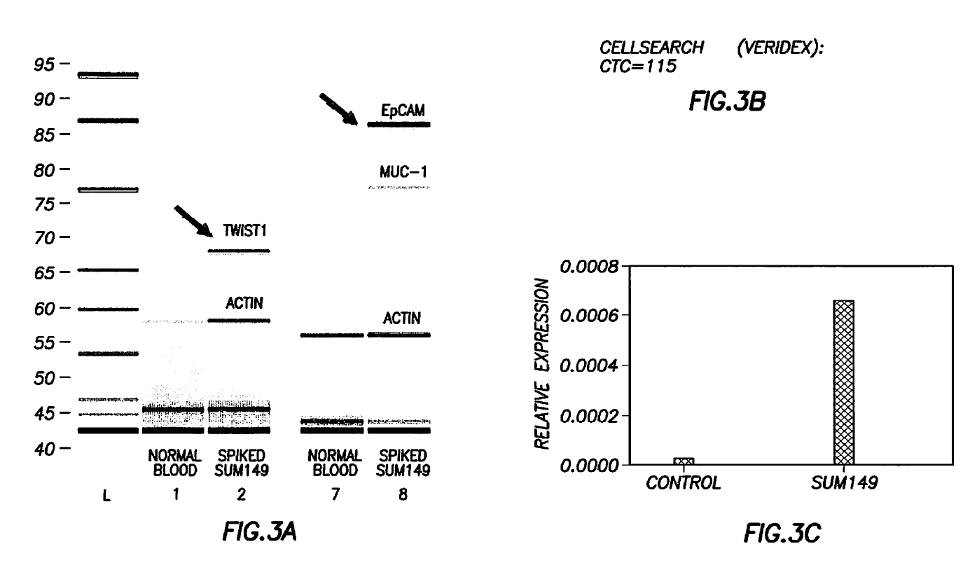

These analyses revealed that epithelial cells from HMEC-TWIST were not to be detected by either CellSearch™ or the AdnaTests, whereas epithelial cells from HMEC-Control were detected by both assays (FIGS. 2A and 2B). On the other hand, SUM149 were detected by both CellSearch™ and AdnaTest (FIG. 3A) and as well as the SUM149 AdnaTest EMT kit (FIG. 3B). These results suggested that while both CellSearch™ and AdnaTest were able to detect CTCs based on the expression of EpCAM, both these assays were not capable of detecting epithelial cells that have undergone “complete EMT”. Therefore, we modified our screen and spiked HMEC expressing Twist and SUM149 cells into healthy donor PB samples and then subjected these samples to our qRT-PCR based detection method as described earlier. Using this approach, we were able to detect HMEC-TWIST (FIG. 2C) and SUM149 cells (FIG. 3C) spiked in the PB samples of healthy donors by following TWIST1 expression in the CD45-depleted PBMC. In addition, we were also able to detect the expression of Snail and Slug, two EMT inducers, in CD45-depleted PBMC that were spiked with SUM149 (data not shown).

Expression of EMT-Related Gene Transcripts TWIST1, SNAIL1, SLUG, ZEB1, and FOXC2 in CD45 Depleted PBMC from Healthy Donors

SLUG mRNA was not detected in any of the replicates from 30 healthy donors investigated. TWIST1, SNAIL1, ZEB1 and FOXC2 were detected in 23.3%, 86.7%, 93.3% and 93.3% of healthy donors, respectively. The relative concentrations of TWIST1, SNAIL1, ZEB1 and FOXC2 mRNA in the healthy donor population was calculated, and the highest value of 2.0×10⁻⁴, 1×10⁻², 2.2×10⁻² and 2.1×10⁻² for TWIST1, SNAIL1, ZEB1 and FOXC2, respectively was used as a cut-off for normal mRNA level for further analysis.

### Relative Quantification of TWIST1, SNAIL1, SLUG, ZEB1 and FOXC2 Gene Transcripts in CD45 Depleted PBMC in Breast Cancer Patients

Patient characteristics are shown in Table 1 below. Median age at the time of processing was 54 years (range: 34-72 years). Among the samples from 52 patients with early breast cancer analyzed in study A, 30 (57.7%) samples expressed TWIST1, 47 (90.4%) SNAIL1, 6 (11.5%) SLUG, and 52 (100%) ZEB1, respectively. All 52 patient samples expressed FOXC2. TWIST1 was more commonly expressed in breast cancer patient compared to healthy donors (57.7% vs. 23.3%; p=0.003). Furthermore, 1 (1.9%) patient had overexpression of TWIST1 compared to cut-off level, 0 (0%) SNAIL1, 6 (11.5%) SLUG, 0 (0%) ZEB1 and 1 (3.2%) FOXC2, respectively. Overexpression of at least one of the EMT genes was detected in 8 (15.4%) patients. There was no overlap between overexpression of EMT genes in these patient samples.

TABLE 1
Patient Characteristics (n = 75)

| Variable | N | % |
|---|---|---|
| Non-metastatic breast tumors | | |
| T stage | | |
| 1 | 24 | 40.00 |
| 2 | 21 | 35.00 |
| 3 | 7 | 11.67 |
| 4 | 8 | 13.33 |
| Node status | | |
| N0 | 29 | 48.33 |
| ≧ N1 | 31 | 51.67 |
| Metastatic breast cancer | 15 | 20.00 |
| Histology | | |
| Infiltrative ductal carcinoma | 59 | 78.67 |
| Infiltrative lobular carcinoma | 8 | 10.67 |
| Other | 8 | 10.67 |
| ER/PR status | | |
| Positive for either | 53 | 70.67 |
| Negative for both | 22 | 29.33 |
| Her/Neu status | | |
| Amplified | 12 | 16.00 |
| Non-amplified | 63 | 84.00 |
| High grade | 30 | 40.00 |
| Triple negative | 18 | 24.00 |
| Inflammatory breast cancer | 19 | 25.33 |
| Neoadjuvant therapy | 17 | 22.67 |
| Pathologic complete response | 5 | 29.41 |

Among the samples from 23 patients with IBC and/or metastatic breast cancer analyzed in study B, 15 (65.2%) expressed TWIST1, 21 (91.3%) SNAIL1, 1 (4.3%) SLUG, 22 FOXC2 (95.7%) and 22 (95.70%) ZEB1, respectively. Again, we observed that TWIST1 was more commonly expressed in breast cancer patients compared to healthy donors (65.2% vs. 23.3%; p=0.004). Furthermore, 2 (8.7%) patients had overexpression of TWIST1 compared to cut-off level, 0 (0%) SNAIL1, 1 (4.3%) SLUG, 0 (0%) ZEB1 and 4 (17.4%) FOXC2. Overexpression of at least one EMT gene was detected in 5 (21.7%) patients. In two samples, there was overlap between overexpression of EMT genes (TWIST1 and FOXC2 or TWIST1 and SLUG).

### EMT Genes Overexpression in Relation to Clinicopathological Parameters

Overexpression of TWIST1, SNAIL1, SLUG, ZEB1 and FOXC2 in relation to various clinicopathological characteristics are given in Table 2 below. We observed that, after neoadjuvant chemotherapy, patients had a higher expression of EMT genes compared to patients with no prior therapy (41.18 vs. 10.34; p=0.007). This observation was not accompanied with association between EMT genes expression and tumor size. Non-metastatic breast cancer patients with positive auxiliary lymph nodes had higher EMT gene expression compared to patients with no lymph node involvement (29.03 vs. 6.90; p=0.04). Of the 17 patients who received neoadjuvant therapy, 5 (29.41%) achieved pathologic complete response (pCR). Whereas only 1 of 5 patients with pCR had overexpression of EMT genes, 6 of 6 patients without pCR had overexpression of EMT genes.

EMT Genes Overexpression Relative to CTCs Count Measured by CellSearch™ and AdnaTest

CTC measurement was available for 57 (76%) patients. Overall median CTC count was 0 (range: 0-750). Seventeen (28.8%) patients had detectable CTC by CellSearch™, while in 30 patients, CTC were not detected. Patients with a positive CTC level either by CellSearch™ or AdnaTest had a higher probability of overexpression EMT genes compared to patients with negative CTC test as shown in Table 2 below.

TABLE 2
The association between any EMT gene overexpression and patient
characteristics

| | Normal | | | | |
|---|---|---|---|---|---|
| | EMT genes | | EMT genes | | |
| | expression | | over-express | | |
| Variable | N | % | N | % | p-value |
| CTC ADNA test select/detect | | | | | |
| (n = 42) | | | | | |
| Negative | 24 | 88.89 | 3 | 11.11 | 0.23 |
| Positive | 11 | 73.33 | 4 | 26.27 | |
| CTC CellSearch ™ (n = 57) | | | | | |
| Negative | 30 | 83.33 | 6 | 16.67 | 0.99 |
| Positive | 17 | 80.95 | 4 | 19.05 | |
| CTC ADNA test/CellSearch ™ | | | | | |
| (n = 57) | | | | | |
| Positive for either | 27 | 79.41 | 7 | 20.59 | 0.29 |
| Negative for both | 21 | 91.30 | 2 | 8.70 | |
| T stage (non-metastatic BC) | | | | | |
| T1 | 19 | 86.36 | 3 | 13.64 | 0.52 |
| ≧ T2 | 30 | 78.95 | 8 | 21.05 | |
| Node status (non-metastatic | | | | | |
| BC) | | | | | |
| N0 | 27 | 93.10 | 2 | 6.90 | 0.04 |
| ≧ N1 | 22 | 70.97 | 9 | 29.03 | |
| Metastatic breast cancer | 13 | 86.67 | 2 | 13.33 | 0.73 |
| Non-metastatic breast cancer | 49 | 81.67 | 11 | 18.33 | |
| Histology | | | | | |
| Infiltrative ductal carcinoma | 48 | 81.36 | 11 | 16.31 | 0.72 |
| Other | 14 | 87.50 | 2 | 12.50 | |
| ER/PR status | | | | | |
| Positive for either | 43 | 81.13 | 10 | 18.87 | 0.74 |
| Negative for both | 19 | 86.36 | 3 | 13.64 | |
| Her/Neu status | | | | | |
| Positive | 11 | 91.67 | 1 | 8.33 | 0.46 |
| Negative | 51 | 80.95 | 12 | 19.05 | |
| High grade | 30 | 81.08 | 7 | 18.92 | 0.77 |
| Low/intermediate grade | 32 | 84.21 | 6 | 15.79 | |
| Triple negative | 47 | 82.46 | 10 | 17.54 | 0.99 |
| Non-triple negative | 15 | 83.33 | 3 | 16.67 | |
| Inflammatory breast cancer | 15 | 78.95 | 4 | 21.05 | 0.73 |
| Non-inflammatory breast cancer | 47 | 83.93 | 9 | 16.07 | |
| Neoadjuvant therapy | 10 | 58.82 | 7 | 41.18 | 0.007 |
| No therapy | 52 | 89.66 | 6 | 10.34 | |
| Pathologic complete response | 4 | 80.00 | 1 | 20.00 | 0.34 |
| (pCR) | 6 | 50.00 | 6 | 50.00 | |
| Other than pCR | | | | | |

We claim:

1. An assay useful to detect breast cancer in a patient and characterized by the detection of circulating tumor cells undergoing epithelial mesenchymal transition in peripheral blood wherein CTCs which do not have an epithelial antigen and through quantitative reverse transcription polymerase chain reaction (“RT-PCR”), the expression of three or more of EMT gene transcripts are identified, the EMT gene transcripts being selected from the group consisting of: TWIST1, SNAIL1, SLUG, ZEB1 and FOXC2.

2. The assay of claim 1, wherein CD45-depleted peripheral blood mononuclear cells are used to detect the expression of EMT genes in the patient.

3. The assay of claim 1, wherein the assay is used in combination with other EpCAM-based selection methods.

4. An assay that detects breast cancer in the peripheral blood patient, wherein the peripheral blood of the patient contains CTCs lacking an epithelial antigen, gene or marker and the expression of at least three EMT gene transcripts selected from the group of TWIST1, SNAIL1, SLUG, ZEB1 and FOXC2 are identified and breast cancer is diagnosed without a correlation to CTC count.

5. A method of diagnosing breast cancer in a human or animal, the method comprising the steps of: a) extracting CTCs from blood plasma or serum obtained from a human or animal; b) identifying CTCs without an epithelial antigen; and c) detecting the expression of at least three EMT gene transcripts using RT-PCR, wherein a breast cancer diagnosis can be confirmed when the blood of a human or animal contains CTCs without an epithelial antigen and the expression of at least three EMT gene transcripts.
